(19) United States
(12) Patent Application Publication
(10) Pub. No.: US 2025/0268755 A1
(43) Pub. Date: Aug. 28, 2025

(54) HANDHELD FIBER PROBE FOR LASER TREATMENT OF MEDICAL TISSUE, IN PARTICULAR FOR TREATMENT OF GLAUCOMA

(71) Applicant: Lab-on-Fiber GmbH, Sonnefeld (DE)

(72) Inventors: Ruzin AGANOGLU, Sonnefeld (DE); Stefan KUFNER, Sonnefeld (DE)

(21) Appl. No.: 19/205,017

(22) Filed: May 12, 2025

Related U.S. Application Data
(63) Continuation of application No. PCT/EP2024/085175, filed on Dec. 6, 2024.

(30) Foreign Application Priority Data
Dec. 12, 2023 (DE) 10 2023 212 552.8

(51) Int. Cl. A61F 9/008

(52) U.S. Cl. CPC A61F 9/008; A61F 2009/00891

(57) ABSTRACT

A handheld fiber probe provides laser treatment of medical tissue. In the assembled state, the fiber probe contains a housing having a front end with an exit point for laser light, and a fiber optic cable having an optical fiber with a fiber termination. The fiber termination of the optical fiber is disposed in the housing. Lenses are provided including a front lens and a rear lens being adjusted relative to one another by a user, so that a laser beam can be varied by varying a distance between the front and rear lenses. The rear lens are a monolithic part of the fiber termination and is formed by fusing the fiber termination.

### CROSS-REFERENCE TO RELATED APPLICATION

This application is a continuation, under 35 U.S.C. § 120, of copending International Patent Application PCT/EP2024/085175, filed Dec. 6, 2024, which designated the United States; this application also claims the priority, under 35 U.S.C. § 119, of German Patent Application DE 10 2023 212 552.8, filed Dec. 12, 2023; the prior applications are herewith incorporated by reference in their entireties.

### FIELD AND BACKGROUND OF THE INVENTION

The invention relates to a handheld fiber probe for laser treatment of medical tissue, in particular for the treatment of glaucoma. The handheld fiber probe which, at least in the assembled state, has a fiber optic cable, with a housing which has a front end with an exit point for laser light.

Such a fiber probe can be found, for example, in published, non-prosecuted German patent application DE 10 2017 104 673 A1 or European patent EP 3 478 205 B1, corresponding to U.S. Pat. Nos. 10,758,118, 11,576,569 and 12,167,891.

Elevated intraocular pressure (IOP) is a major risk factor for the development of glaucoma. With the aid of therapeutic laser treatment using such fiber probes, the intraocular pressure is reduced, for example by limiting the formation of aqueous humor and/or by improving the drainage of the aqueous humor.

During treatment with laser light, specific parts of the eye tissues are treated. The fiber probe is placed on the eye with a front distal contact end.

Published, non-prosecuted German patent application DE 10 2017 104 673 A1 describes an attachment that can be screwed onto a handle to which a fiber optic cable is connected. A hole is formed within the attachment into which an optical fiber (optical waveguide fiber) of the fiber optic cable can be inserted. The hole ends inside the attachment, so that the front contact end is sealed. This prevents liquid from penetrating into the annular space between the optical fiber and the hole during laser treatment due to capillary action and thereby contaminating the attachment. Furthermore, an optical element for beam shaping and, for example, for focusing the laser beam is attached to the front surface of the front contact end.

European patent EP 3 478 205 B1 also discloses a handheld fiber probe in which a disposable attachment can be attached to a handle in which an optical fiber is arranged, which fiber probe protrudes from the front contact end and interacts with an optical element in the handle to focus the laser light.

The following problems, among others, have emerged in medical practice when using such fiber probes, depending on the type of fiber probe involved.

Since the fiber probe is placed on the eye with a certain amount of pressure during treatment and/or a large amount of energy is applied via the laser light, the treatment is often perceived as painful by patients. Consequently, such treatments are often carried out under anesthesia.

Bleeding may occur during treatment, which may lead to contamination of the attachment due to capillary action, for example, which may limit the effectiveness of the applied laser light during treatment. For example, the transmission is reduced and thermal heating occurs due to undesired absorption of laser light in the attachment.

Due to differing anatomies among patients, the guidance of the handheld fiber probe by the medical staff requires experienced medical personnel. In some cases, medical personnel manually place an intermediate layer, such as a thin silicone pad, between the distal contact end of the fiber probe and the eye.

### SUMMARY OF THE INVENTION

Based on this, it is the object of the invention to provide a handheld fiber probe for laser treatment of medical tissue, in particular for the treatment of glaucoma, with which at least some of the problems described above are at least reduced.

The object is achieved according to the invention by a handheld fiber probe for laser treatment of medical tissue and, in particular, for the treatment of glaucoma. A fiber optic cable is connected to the fiber probe, at least when in the assembled state. The fiber probe has a housing having a front end, which is also referred to below as the distal contact end, which is designed to rest on the eye. For this purpose, the contact end has a concavely curved surface that is fitted to the human eye. An exit point for laser light is formed at this front contact end. Furthermore, the fiber probe has optics for beam shaping, in particular for focusing the laser light. These optics have at least two lenses, namely a front and a rear lens, which can be adjusted relative to one another by the user, enabling the laser beam to be varied, meaning that beam shaping, in particular focusing, can be achieved by varying a distance between the two lenses.

What is particularly noteworthy in this design is the variation of the focus and hence the alteration of a focal point of the laser beam by the medical staff themselves. This enables the doctor to change the focus during treatment and adapt it specifically to the anatomy of each patient.

The focus is varied by changing the distance between the two lenses. An adjustment mechanism is generally configured for this purpose which can be operated manually by the respective operator (doctor, medical staff) in order to vary the distance.

The ability of the doctor to vary the focus during treatment enables the doctor to focus the laser energy in a targeted and highly precise manner on a specific treatment site. Manual variation of distance, for example by means of the sometimes practiced arrangement of an intermediate layer (silicone pad) between the front end and the eye, is therefore no longer necessary. This improves the operability of the fiber probe. The improved focusing also increases efficiency and reduces the total laser energy input. This results in a much gentler and less painful treatment. This also makes it possible, in particular, to omit anesthesia.

The two lenses generally create adjustable optics that enable the focus of the laser beam to be varied. The lens system used for this purpose is constituted in particular by the two (individual) lenses described herein.

The fiber probe is configured to be guided manually by the user (doctor) during treatment and to be placed externally on a part of the body, in particular on the eye, with the contact end facing forward.

The housing contains a fiber termination of an optical fiber that is designed to guide the laser light. A rear lens is formed at one end of this optical fiber. The rear lens is therefore a part of the optical fiber (the fiber termination) itself and is therefore a monolithic component of the optical fiber. The rear lens is created by fusing/melting the fiber end so that the fiber material is formed into a lens shape, in particular a spherical shape, at the end. The optical fiber is particularly a glass fiber.

The other, front lens is preferably formed at the exit point.

Both lenses are preferably embodied as a spherical lens. They are made primarily of quartz glass. Accordingly, a glass fiber is used for the fiber termination.

In a preferred refinement, a guide channel formed by a free space is formed at least between the two lenses, with at least one of the lenses being adjustable within this guide channel in order to vary the distance.

The distance between the two lenses is preferably between 2 mm and 5 mm and in particular 3.5 mm. Preferably, the distance can be varied by a (maximum) difference in distance. This difference in distance between the two lenses is preferably in the range of a few millimeters and in particular in the range of less than 3.5 mm. The preferred distance between the two lenses depends in particular on the diameter of the optical fiber. The smaller the diameter, the shorter the distance.

The optical fiber is preferably embodied as a multimode fiber and therefore has a substantially larger diameter than a single-mode fiber.

The optical fiber preferably has a diameter of a few or several 100 μm up to, for example, 1000 μm. Typical values are 200 μm, 400 μm, or 600 μm. In particular, the optical fiber generally has a diameter in the range of from 200 μm to 600 μm. The distance values given above apply in particular to a fiber with a diameter of 600 μm. Insofar as diameter is mentioned herein, this is to be understood in particular to mean a core diameter of a fiber core of a fiber, the fiber being formed by the fiber core and a fiber cladding surrounding the same. The total diameter of the fiber including the fiber cladding is typically 100 μm to 200 μm greater than the core diameter and is, for example, 300 μm for a fiber with a fiber core of 200 μm and 750 μm for a fiber with a fiber core of 600 μm.

Especially by virtue of the design as a multimode fiber with a comparatively large diameter, the requirements for the entire optics are not excessively high, enabling the fiber probe as well as an associated light source (laser) to be designed cost-effectively. This is particularly true in connection with the monolithic formation of the rear lens at the fiber end, in particular by melting the fiber end.

In general, the fiber probe is associated with a laser to which the fiber probe is connected via the optical fiber and which generates the laser light. The laser is preferably a diode laser in which the laser light is generated in a simple manner by one and preferably only one single laser diode. Alternatively, diode lasers with multiple wavelengths emitted simultaneously or sequentially are also used.

The wavelength of the laser light is in the range from 800 nm to 1000 nm, for example. A diode laser with a wavelength of 810 nm is preferably used.

The light output of the laser is typically in the range of a few watts, for example in the range from 1 W to 5 W and particularly in the range from 1.5 W to 3 W.

A continuous laser beam is preferably provided during operation. Alternatively, a pulsed laser beam with comparatively long pulses in the ms range (1 ms to 100 ms or more) can also be set. Unlike in complex optical systems in which a pulsed laser beam is used, e.g., with very short pulses in the nanosecond range or even shorter pulse durations, such as in the femtosecond range, the required optical components can have a comparatively simple and inexpensive design.

The geometric conditions, for example the distance between the lenses, the diameter and/or the curvature of the lenses, are preferably selected such that—for a given wavelength of the laser light (for example, 810 nm)—the light emerging from the rear lens enters directly at least largely (greater than 90%, preferably greater than 95% of the intensity) into the front lens without being reflected on the channel walls of the guide channel.

In a preferred embodiment, the front lens is fixed at the exit point, and the rear lens is arranged so as to be displaceable within the guide channel. In particular, the front lens is attached directly to a front end face, particularly to an opening formed by the guide channel.

In a preferred embodiment, the two lenses have different lens diameters. Specifically, the rear lens has a smaller lens diameter than the front lens.

Preferably, the guide channel has a constant channel diameter over its length, at least within a longitudinal section in which the two lenses are arranged.

The lens diameter of the rear lens is adapted to the channel diameter of the guide channel such that displacement is possible. Specifically, a tolerance gap is formed between the rear lens and the guide channel.

The front lens preferably has a diameter of greater than 0.8 mm and, in particular, a diameter in the range from 1 mm to 3 mm.

The rear lens, on the other hand, has a smaller diameter, for example in the range from 0.2 mm to 1 mm. The diameter of the rear lens is preferably smaller than that of the front lens by a factor of 1.1 to 3.

The front lens preferably has a diameter which is larger than the diameter of the guide channel. The front lens is therefore placed on the opening of the guide channel from the outside and rests on one edge of this opening. The diameter ratios between the lenses selected above therefore allow one lens to be moved in the guide channel while the other lens can be positioned at the opening of the guide channel.

The front lens is preferably the frontmost lens of the fiber probe, meaning that the light for the eye to be treated emerges from this front lens. The frontmost lens preferably forms the frontmost portion of the fiber probe. During treatment, the front lens is preferably placed on the eye to be treated. This means that the fiber probe is designed for direct placement of the front lens onto the eye to be treated. The direct placement of the front lens on the eye to be treated is not prevented, for example, by bothersome housing structures or an additional attachment.

In a preferred embodiment, an opening, specifically a channel opening and in particular the previously described opening of the guide channel, is generally formed at the front end, which opening is sealed by the front lens. For this purpose, the diameter of the front lens is larger than the diameter of the opening, as explained previously. The front lens therefore rests sealingly on one edge of this opening.

This aspect of the sealing closure of such a channel opening at the front end of the fiber probe housing is considered to be an independent inventive idea independent of the design with the adjustable lenses.

In a preferred embodiment, the front lens is integrally connected to the opening and specifically to this opening edge. This is done, for example, using an adhesive and preferably by fusion. The front end is made in particular of glass, more particularly quartz glass, so that a material-locking connection can be achieved through appropriate thermal treatment by fusing between the front end and the front lens. This ensures reliable sealing. There is therefore no risk of contamination by liquid penetrating the channel, especially as a result of capillary action.

In a preferred embodiment, the fiber probe has an adjustment mechanism that can be operated by the user and, in particular, manually, by means of which the distance between the two lenses can be varied. With the aid of the adjustment mechanism, the distance is changed by mechanically moving the lenses relative to one another.

For this purpose, the adjustment mechanism preferably has a manually operable adjusting element so that it can be operated by hand by medical personnel. The adjusting element can be, for example, a sliding element, a rotating element, or an adjustment wheel. The adjusting element is arranged in particular on a cladding side of the housing of the fiber probe or is itself formed by a cladding region and thus by a portion of the housing.

In a preferred development, in order to form the adjustment mechanism, it has an actuating part which is adjustable in a longitudinal direction and which is connected to the at least one lens for adjustment thereof. The adjustment movement of this actuating part is initiated via the control element.

Preferably, the actuating part is a housing part which is adjustable relative to another housing part.

In one embodiment, the adjustment is carried out by a rotary movement, for example, in particular of the actuating part itself, in particular in such a way that the actuating part is rotatably mounted on an additional part, for example on the additional housing part, by means of a thread and is thus displaceable in the longitudinal direction relative to this additional part.

In a preferred embodiment, the fiber termination is held by the actuating part and is securely connected thereto in an integral manner, for example by gluing. The connection is made at a distance from the rear lens. The adjustment of the rear lens is therefore carried out indirectly via an adjustment of the fiber termination. This is structurally easy to implement.

The fiber termination has in particular a rear portion which is held by the actuating part and in particular rests therein. The actuating part has in particular a channel which is in particular a part or an extension of the previously mentioned guide channel through which the fiber termination is guided. The rear portion of the fiber termination, for example, fits precisely and in particular frictionally or integrally into this channel and is thus fastened in the actuating part.

The rear portion is preferably surrounded by a cladding, for example a protective tube, which is secured in the adjusting element with sufficient holding force—for example by frictional engagement or material bond—so that when the adjusting element is adjusted, the fiber termination and with it the rear lens are displaced in the longitudinal direction relative to the front lens.

In one expedient embodiment, the housing has a rear housing part which is embodied in particular as a handle and is therefore configured to be held in the doctor's hand during operation.

Furthermore, the housing has a front housing part which is preferably connected in a reversibly detachable manner to the rear housing part. This front housing part is therefore a replaceable tip or a replaceable attachment.

The housing preferably consists of these two housing parts, i.e., the handle and the interchangeable attachment.

The previously described guide channel extends from the rear housing part into the front housing part and runs to the exit point. The guide channel is embodied in particular as a continuous guide channel which extends from a rear, proximal opening of the housing to the front distal opening at the front end, at which the guide channel is sealed in particular by the front lens.

The rear housing part preferably has a continuous channel which is a portion or an extension of the guide channel through which the fiber termination can be pushed.

In one expedient embodiment, the front housing part is made of a transparent material and in particular of glass or quartz glass or alternatively of a polymer.

As an alternative to the transparent design, a non-transparent design of the front housing part is used. In that case, it is made of metal or ceramic, for example.

According to an optional configuration, a coating is additionally applied in the vicinity of the guide channel in order to reflect laser light on the channel walls. Such reflective coatings are useful in lenses, for example, in order to reduce Fresnel reflections and thus increase transmission.

The rear housing part, on the other hand, is preferably made of a non-transparent material, for example of plastic, but preferably of metal, for example of aluminum, in particular a colored and, for example, black, blue, or red anodized aluminum.

The fiber probe is, in particular, a preconfigured disposable or reusable part in which the fiber optic cable is already connected. The fiber optic cable has at its proximal end a connecting part, for example, in particular a plug part for connection, in particular for plug-in connection, to an optical component. The optical component is, for example, a laser or another fiber optic cable, quasi as an extension for connecting to a laser.

The fiber probe described herein makes it possible to design a cost-effective fiber probe with an adjustable, comparatively wide focus while maintaining a simple and cost-effective construction.

Preferably, a set of multiple differently designed fiber probes is provided which differ with regard to the diameter of the front lens and in particular only with regard to the diameter of the front lens. The different diameters with otherwise identical construction make different focal lengths available, so that a wide range of applications is covered for different anatomies, e.g., in children and adults, via the multiple fiber probes.

Other features which are considered as characteristic for the invention are set forth in the appended claims.

Although the invention is illustrated and described herein as embodied in a handheld fiber probe for laser treatment of medical tissue, in particular for treatment of glaucoma, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 is a diagrammatic, partial side sectional view of a fiber probe;

FIGS. 2A, 2B are schematic diagrams explaining a variable adjustment of a focus of a laser beam through variation of a distance between two spaced lenses; and

FIGS. 3A-3C are illustrations showing the fiber probe with an additional optical attachment element in different variants.

### DETAILED DESCRIPTION OF THE INVENTION

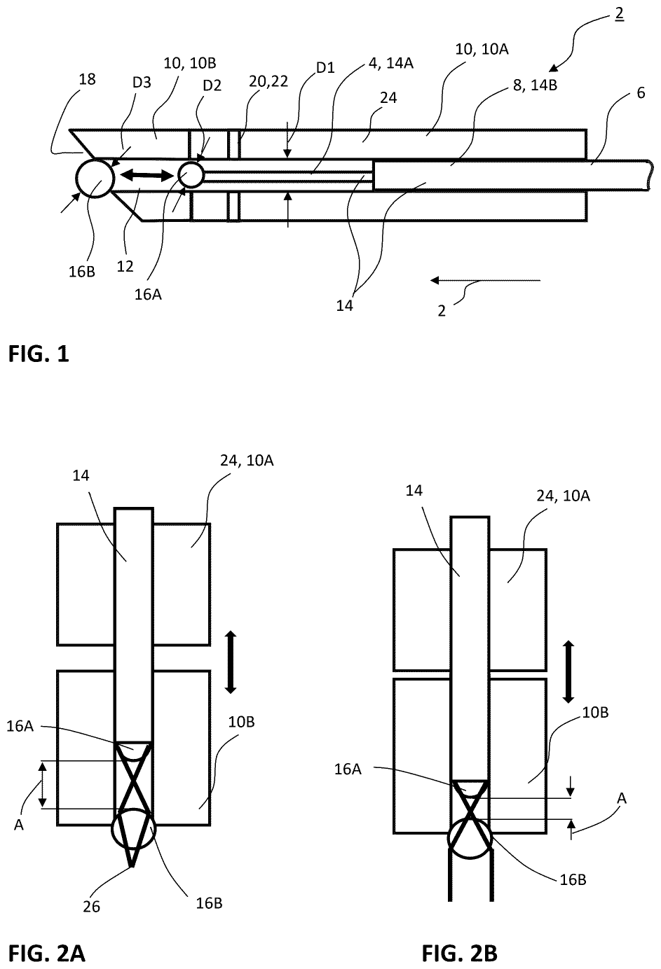

Referring now to the figures of the drawings in detail and first, particularly to FIG. 1 thereof, there is shown a manually operable fiber probe 2 used for the therapeutic treatment of medical tissue and in particular for the treatment of glaucoma using laser light. Laser light has a wavelength of 810 nm, for example. The laser light is provided by a laser (not shown here), in particular a diode laser, and is coupled into an optical fiber 4 (optical waveguide fiber) that forms part of a fiber optic cable 6. The fiber optic cable 6 has in particular a cladding 8 which is embodied, for example, as a hose in which the optical fiber 4 is guided.

A multimode fiber is used as the optical fiber whose diameter (core diameter) is in the range between 200 μm and 600 μm. The fiber preferably has a numerical aperture in the range from 0.2 to 0.5.

The fiber probe 2 is a preconfigured component in which the fiber optic cable 6 is already part of the fiber probe 2. It therefore only needs to be connected to a laser with a rear end of the fiber optic cable 6.

The fiber probe 2 has a two-part housing 10 in the exemplary embodiment with a rear housing part which forms a handle 10A. A front housing part is attached to this handle 10A, for example by means of a screw connection, and is generally preferably reversibly replaceable. This front housing part therefore constitutes an attachment 10B. The housing 10 extends in a longitudinal direction L from the handle 10A to the attachment 10B.

The two housing parts 10A, 10B form an internal, continuous channel which is referred to as the guide channel 12. In the exemplary embodiment, this has a constant diameter D1. A fiber termination 14 of the optical fiber 4 extends into this guide channel 12 and is subdivided within the housing 10 into a rear portion 14A and a front portion 14B. In the rear portion 10A, the fiber 4 is still surrounded by the cladding 8, whereas it is exposed in the front portion 14B.

At the front fiber end of the fiber termination 14, as viewed in the longitudinal direction L, a particularly spherical rear lens 16A is formed on the end of the fiber 4. This is formed in particular by fusing the fiber 4. The rear lens 16A has a lens diameter D2.

The housing 10, in particular the attachment 10B, has a front end 18 (distal contact end) as viewed in the longitudinal direction L. The frontal surface formed there is typically adapted to the curvature of the human eye and is embodied in particular as a concavely curved frontal surface which is also oriented obliquely to the longitudinal direction L.

The guide channel 12 leads at this front end 18 to an opening which is sealed by a front lens 16B. The front lens 16B also defines an exit point for the laser light. Preferably, both the attachment 10B and the front lens 16B are made of glass, and the front lens 16B is integrally bonded to the attachment 10B by fusion. As a result, the front lens 16B reliably seals the opening and hence the guide channel 12 and prevents the penetration of dirt particles or liquid, which could lead to contamination and thus in particular to an impairment of the propagation of the laser light during operation.

The two lenses 16A, 16B are adjustable relative to one another in the longitudinal direction L, so that a distance A between the two lenses 16A, 16B can be varied. The focus of the laser light can be changed during operation by varying the distance.

The front lens 16B is arranged so as to be stationary relative to the housing 10, and the rear lens 16A is arranged so as to be displaceable within at least a portion of the guide channel 12. The rear lens 16A together with the fiber termination 14 is displaceable relative to the front lens 16B.

To vary the distance A, the fiber probe 2 has an adjustment mechanism 20, which is only illustrated in a very simplified manner in the figures. This adjustment mechanism has a hand-operated adjusting element 22 which can be manually operated by the operator. The adjusting element 22 causes the relative adjustment of the two lenses 16A, 16B to one another.

The adjusting element 22 is arranged in particular on the outside of the housing, i.e., on the cladding, or is formed by the cladding itself. For example, it is an adjustment wheel which can be operated with the fingers by a rotating movement.

The adjustment mechanism 20 further comprises an actuating part 24 which, in the exemplary embodiment, is formed in particular by the handle 10A and thus by a housing part. The actuating part 24 is generally connected to the displaceable rear lens 16A. Specifically, it is expediently fastened to the cladding 8 of the fiber termination 14, for example by frictional engagement and in particular by material bonding, for example by gluing, so that the entire fiber termination 14 is displaced by an adjusting movement of the actuating part 24 in or counter to the longitudinal direction L.

In the preferred embodiment, in which the actuating part 24 is embodied as a housing part, the two housing parts 10A, 10B are displaceable relative to one another in the longitudinal direction L.

The adjustment of the actuating part 24 is effected by the adjusting element 22, which, for example, via a worm gear, converts a rotary movement into a longitudinal movement of the adjusting element 22. Other mechanical actuation devices are also possible in principle. For example, a separate actuating part 24 can be arranged inside the housing 10 which grips the fiber termination 14, in particular in the rear portion 14A. The actuating part 24 is designed, for example, in the manner of a slide which rests in a portion of the guide channel 16.

Especially in a variant in which the housing parts 10A, 10B are offset relative to one another in the longitudinal direction L, the adjusting element 22 is formed, for example, by a cladding region or a grip recess on the handle 10A and the two housing parts 10A, 10B are connected to one another by means of a thread, so that the desired change in distance is achieved by rotating the two housing parts 10A, 10B relative to one another.

The two housing parts are, for example, two portions of the handle 10A that are connected to one another by means of a thread.

The mode of operation will be explained with reference to FIG. 2A and FIG. 2B: In the two figures, two housing parts 10A, 10B are sketched as examples which have a distance between them that can be varied in the longitudinal direction L. This changes the distance A between the two lenses 16A, 16B.

In the exemplary embodiment, both lenses 16A, 16B are convexly curved. They are therefore embodied as converging lenses due to their convex curved shape and in particular lens shape or spherical shape. Possible beam paths of the laser light depending on the distance A are shown in the two figures. In the situation according to FIG. 2A, a particular maximum distance A is set in which a focal point 26 of the laser beam generated by the two lenses is set as close as possible in front of the front lens 16B. As the distance A becomes smaller, the laser beam widens or the focal point 26 shifts forward in the longitudinal direction L.

As an alternative to this illustrated variant, in which the front lens 16B is a simple lens, the front lens 16B can also be embodied as a complex lens system and/or as an objective. In that case, concave lens surfaces can then also be employed.

However, the preferred variant is the design with two simple individual lenses shown in FIG. 1 and FIG. 2A and FIG. 2B.

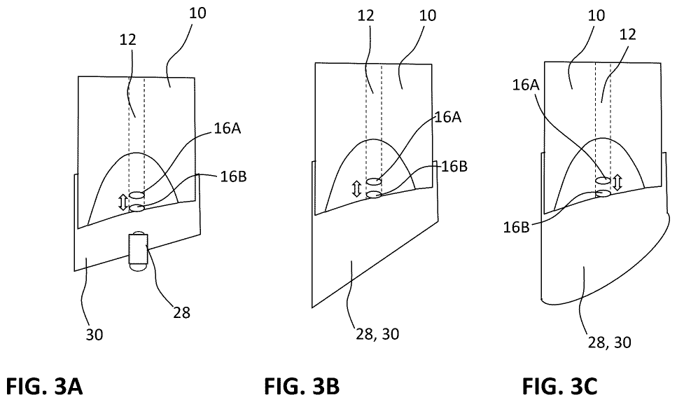

Finally, it is also possible to arrange additional optics 28 as part of an attachment element 30 or directly as the attachment element 30 itself on the housing 10, as shown in FIGS. 3A to 3B. These additional optics 28 is therefore generally arranged in the beam direction downstream from the two lenses 16A, 16B which are adjustable relative to one another. In alternative, preferred variants, however, additional optics 28 and/or attachment elements 30 are omitted.

In FIGS. 3A to 3B, the housing 10 is shown in a highly simplified manner, without explicitly illustrating the two housing parts 10A, 10B and the adjustment mechanism 20 for the relative adjustment of the two lenses 16A, 16B to one another. The attachment element 30 is in particular mounted so as to be stationary relative to the front housing part 10B. For example, it is attached to this front housing part 10B. Alternatively, it can also be stationary relative to the rear housing part 10A.

For this purpose, the additional attachment element 30 has, for example, a sleeve-shaped portion with which it is attached to the housing 10. In particular, the attachment element 30 is slipped onto the housing 10 and is appropriately secured and held there. The attachment element 30 is preferably reversibly interchangeable, so that the fiber probe 2 can be used without or with different attachment elements 30 as needed.

The attachment element 30 is designed differently depending on the area of application and requirements. According to FIG. 3A, a separate element is mounted in or on the attachment element 30 as the optics 28, which is embodied for example as an objective with multiple lenses. The optics 28 are generally used for further beam shaping, e.g., focusing or expanding the laser beam.

According to FIG. 3B and FIG. 3C as well, the attachment element 30 itself forms optics 28. In this case, the attachment element 30 as such forms a light-conducting element.

This is designed in the manner of a prism as shown in FIG. 3B. This is used in particular for beam deflection.

According to FIG. 3C, the attachment element 30 itself—in particular in addition to a prismatic design for deflection analogous to FIG. 3B—forms a lens which is used for further beam shaping. In the exemplary embodiment, a convex curved lens for beam focusing is shown.

Preferably, however, such (additional) optics 28 are dispensed with, and the focusing is performed exclusively via the two lenses 16A, 16B. Also, the attachment element 30 preferably does not have optics 28 and is not embodied as a lens. In particular, the attachment element 30 is omitted.

Overall, the adjustment mechanism 20 achieves a special structure of the fiber probe 2 with the adjustable optics formed by the two lenses 16A, 16B, which enables the operator to easily change the focus of the laser beam manually.

The fiber probe 2 is generally used for the treatment of medical (human/animal) tissue by means of laser irradiation, for example on the skin surface. Due to the variable focus, the fiber probe 2 is specifically designed for treating the eye, particularly for the treatment of glaucoma, where the laser beam is used to treat tissue inside the eye.

Special advantages of the fiber probe 2 described herein include:
manually adjustable, variable focus of the laser beam;
reliable sealing of the interior of the fiber probe 2 by the front lens 16B,
and hence the option of multiple use;
due to the focusability, less laser energy is required, resulting in gentler and less painful treatment,
so that anesthesia can be dispensed with.

The following is a summary list of reference numerals and the corresponding structure used in the above description of the invention:
2 fiber probe
4 optical fiber
6 fiber optic cable
8 cladding
10 housing
10A handle
10B attachment
12 guide channel
14 fiber termination
14A rear portion
14B front portion
16A rear lens
16B front lens
18 front end
20 adjustment mechanism
22 adjusting element
24 actuating part
26 focal point
28 additional optics
30 attachment element
A distance
D1 diameter of guide channel
D2 diameter of rear lens
D3 diameter of front lens

1. A handheld fiber probe for laser treatment of medical tissue, which, in an assembled state, comprising:
a housing having a front end with an exit point for laser light;
a fiber optic cable having an optical fiber with a fiber termination, said fiber termination of said optical fiber being disposed in said housing; and
lenses including a front lens and a rear lens being adjustable relative to one another by a user, so that a laser beam is varied by varying a distance between said front and rear lenses, said rear lens being a monolithic part of said fiber termination and is formed by fusing said fiber termination.

2. The handheld fiber probe according to claim 1, further comprising a guide channel formed between said lenses, and at least one of said lenses is adjustable within said guide channel in order to vary the distance.

3. The handheld fiber probe according to claim 1, wherein the distance between said lenses is in a range between 2 mm and 5 mm.

4. The handheld fiber probe according to claim 1, wherein said optical fiber is embodied as a multimode fiber and/or has a diameter in a range of from at least 100 μm to 1000 μm.

5. The handheld fiber probe according to claim 2, wherein said front lens is disposed so as to be stationary at said exit point, and said rear lens is disposed so as to be displaceable within said guide channel.

6. The handheld fiber probe according to claim 1, wherein said front and rear lenses have different lens diameters.

7. The handheld fiber probe according to claim 1, wherein said front lens has a diameter of greater than 0.8 mm.

8. The handheld fiber probe according to claim 7, wherein said rear lens has a diameter which is smaller than said front lens by a factor of 1.1 to 3.

9. The handheld fiber probe according to claim 1, wherein said front end of said housing having an opening formed therein which is sealed by said front lens, said front lens being fixed to sasid opening in a materially bonded manner.

10. The handheld fiber probe according to claim 1, wherein said front and rear lenses are the only said lenses.

11. The handheld fiber probe according to claim 10, wherein said front lens is configured to be placed directly on an eye to be treated.

12. The handheld fiber probe according to claim 1, further comprising an adjustment mechanism by means of which the distance between said front and rear lenses can be varied.

13. The handheld fiber probe according to claim 12, wherein said adjustment mechanism is manually operable and has an adjusting element which is disposed on said housing or is formed by a portion of said housing.

14. The handheld fiber probe according to claim 13, wherein said adjustment mechanism has an actuating part which is adjustable in a longitudinal direction and is connected to said rear lens for adjustment thereof.

15. The handheld fiber probe according to claim 14, wherein said fiber termination is held by said actuating part.

16. The handheld fiber probe according to claim 15, wherein said fiber termination has a rear portion with a cladding, and that said rear portion is fastened to said actuating part.

17. The handheld fiber probe according to claim 14, wherein said housing has a rear housing part which is embodied as a handle and a front housing part with said front end, said front housing part being fastened to said handle as an attachment and made of a transparent material.

18. The handheld fiber probe according to claim 1, further comprising additional optics being attached to said housing in a vicinity of said front end.

19. The handheld fiber probe according to claim 1, wherein the handheld fiber probe is configured to connect to a laser for generating the laser light, the laser is embodied as a diode laser.

20. A fiber probe apparatus, comprising:
a set of a plurality of fiber probes, each embodied according to the handheld fiber probe according to claim 1, wherein said fiber probes differ with respect to a diameter of said front lens.
